(19) United States
(12) Patent Application Publication
(10) Pub. No.: US 2010/0041759 A1
(43) Pub. Date: Feb. 18, 2010

(54) MULTI-PHASE RELEASE POTASSIUM GUAIACOLSULFONATE COMPOSITIONS

(76) Inventors: Glynn Wilson, Duluth, GA (US); Matthew F. Heil, Duluth, GA (US)

Correspondence Address: SHERIDAN ROSS PC, 1560 BROADWAY, SUITE 1200, DENVER, CO, 80202, US

(21) Appl. No.: 12/582,872

(22) Filed: Oct. 21, 2009

Related U.S. Application Data
(63) Continuation of application No. 11/680,355, filed on Feb. 28, 2007.
(60) Provisional application No. 60/828,150, filed on Oct. 4, 2006.

(51) Int. Cl. A61K 31/185; C07C 309/29; A61P 25/00

(52) U.S. Cl. 514/576; 562/76

(57) ABSTRACT

Formulations have been developed administering a guaiacolsulfonate salt, preferably potassium salt, in multi-phases. In a preferred embodiment, the formulation contains potassium guaiacolsulfonate in an immediate release (“IR”) form and a sustained or delayed release (“DR”) form and/or pulsed release (“PR”) form. In another embodiment, the potassium guaiacolsulfonate is released in a gradient, decreasing the side effects associated with rapidly elevated blood levels. In another embodiment, the drug is bound to an ion-exchange resin, which can be suspended in a liquid or incorporated into a matrix for delayed, sustained and/or pulsed release. Dosage unit forms may be tablets, gels, liquids, capsules, beads, microparticles, films or lozenges. Multi-phase delivery can also be achieved through the use of a kit that provides for dosage escalation. The formulations are useful in the treatment of one or more symptoms of coughs, colds, sinusitis and other respiratory illnesses. The formulations are also useful in the treatment or relief of fibromyalgia, pain, and Irritable Bowel Syndrome.

### CROSS-REFERENCE TO RELATED APPLICATIONS

The present application is a continuation of U.S. patent application Ser. No. 11/680,355, filed on Feb. 28, 2007, which in turn claims priority to U.S. Provisional Patent Application No. 60/828,150, filed on Oct. 4, 2006, both of which are incorporated by reference herein in their entireties.

### FIELD OF THE INVENTION

The present invention is generally in the field of multi-phase release potassium guaiacolsulfonate compositions, especially compositions combining immediate release and delayed or sustained release, and compositions for use in dosage loading regimes.

### BACKGROUND

Potassium guaiacolsulfonate is an expectorant used to help loosen and clear mucus. Guaiacolsulfonate has been used in combination with other mucolytic agents, such as guaifenesin, to treat bronchial congestion and coughs associated with the common cold, asthma, and other respiratory illnesses (e.g., sinusitis, pharyngitis, bronchitis).

Potassium guaiacolsulfonate is not in common usage, but has been co-formulated with other compounds for relief of cold symptoms, in both an immediate release and extended release formulation. Modified released formulations containing guaifenesin and potassium guaiacolsulfonate were previously available under the tradenames Humibid.®. LA (tablets containing 600 mg of guaifenesin and 300 mg of potassium guaiacolsulfonate) and Allfen.®. (extended release tablets containing 1000 mg of guaifenesin and 150 mg of potassium guaiacolsulfonate). Typical side effects of guaiacolsulfonate include nausea, headache, dizziness, drowsiness, restlessness, nervousness, and trouble sleeping.

There are a number of benefits in having different drugs available for relief of common cold symptoms. For example, some individuals will not be responsive to a particular drug. Sometimes one drug will work more effectively in combination with a different drug providing the same type of relief.

It is therefore an object of the invention to provide formulations of potassium guaiacolsulfonate, alone or in combination with other drugs, having advantageous release properties or providing enhanced relief from cold symptoms, whilst minimizing one or more side effects.

### SUMMARY OF THE INVENTION

Formulations containing a guaiacolsulfonate salt, preferably the potassium salt, alone or in combination with other pharmaceutically active agents, in multi-phases have been developed. In one embodiment, the formulation contains potassium guaiacolsulfonate in both an immediate release (“IR”) form and a sustained or delayed release (“SR” or “DR”) form and/or a pulsed release (“PR”) form. Typically, delayed release is obtained using an enteric coating applied to a core containing the drug, and then applying a coating of drug over the enteric coating so that this coating of drug is released immediately upon ingestion. Sustained release is usually obtained by mixing excipients which delay dissolution of the drug, and then overcoating this core with an immediate release formulation. In another embodiment, the potassium guaiacolsulfonate is released in a gradient, decreasing the side effects associated with rapidly elevated blood levels of guaiacolsulfonate. In another embodiment, the drug is bound to an ion-exchange resin, which can be suspended in a liquid or incorporated into a matrix for delayed, sustained and/or pulsed release. Suitable dosage unit forms include, but are not limited to, tablets, gels, liquids, capsules, beads, microparticles, films or lozenges.

Multi-phase delivery can also be achieved through the use of a kit that provides for dosage escalation. This kit can be a blister pack or equivalent, wherein the drug is packaged so that a first dosage is taken, followed by sequentially larger dosages. The dosages can be the same in each unit, and instructions provided so that the correct dosage is obtained through the number of units taken and the time of administration, or the dosages may be different, and the units ordered so that the desired dosage administration profile is obtained when the patient takes the units in order as instructed.

Preferred drug combinations include potassium guaiacolsulfonate, phenylephrine and chlorpheniramine; potassium, guaiacolsulfonate; pseudoephedrine and chlorpheniramine; potassium guaiacolsulfonate and dextromethorphan; potassium guaiacolsulfonate, dextromethorphan and phenylephrine; potassium guaiacolsulfonate; dextromethorphan and pseudoephedrine for treatment of colds.

The therapeutic pharmaceutical compositions generally are administered systemically and may be administered in various ways known in the art. In one embodiment, the compositions are provided to the patient by oral administration. Typically, the composition will be provided in tablet or capsule form. The composition can also be provided in a form that can be retained at a mucosal site to further control the speed and extent of potassium guaiacolsulfonate absorption.

In one embodiment, the formulations are for use in the treatment of one or more symptoms associated with the common cold, asthma, and/or other respiratory illnesses such as sinusitis, pharyngitis, and bronchitis. In another embodiment, the formulations are used for the relief of one or more symptoms of fibromyalgia. Formulations for administration to patients with fibromyalgia may also be immediate release or extended release. In additional embodiments, the formulations may be used for the treatment of pain and Irritable Bowel Syndrome.

### DETAILED DESCRIPTION OF THE INVENTION

### I. Definitions

The phrase “alleviating a symptom of a disorder” means reducing or eliminating the severity or the frequency of the symptom or both.

As used herein “potassium guaiacolsulfonate” encompasses potassium guaiacolsulfonate and pharmaceutically acceptable salts thereof; pharmaceutically acceptable, pharmacologically active derivatives of potassium guaiacolsulfonate and their pharmaceutically acceptable salts; and active metabolites of potassium guaiacolsulfonate and their pharmaceutically acceptable salts, unless otherwise noted. It is understood that in some cases dosages of derivatives and metabolites may need to be adjusted.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent, compound formed, for example, from non-toxic inorganic or organic acids. For example, such conventional non-toxic salts include those derived from inorganic acids such as hydrochloric, hydrobromic, sulfuric, sulfamic, phosphoric, and nitric acids; and the salts prepared, from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic acids.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

A “disorder” includes any condition, illness, disease, or infection

Effective amount” or “therapeutically effective amount” means the amount needed for the desired therapeutic effect and includes any additional amount or overage of active ingredient deemed necessary in the formulation to provide the desired amount upon administration.

Immediate Release” or “IR” means the therapeutic pharmaceutical composition is provided in a formulation allowing the active agent to begin acting in a therapeutic manner substantially as soon as the agent becomes available in the body and/or bloodstream of the patient.

A “delayed release dosage form” is one that releases a drug (or drugs) at a time other than promptly after administration.

An “extended release dosage form” is one that allows at least a twofold reduction in dosing frequency as compared to that drug presented as a conventional dosage form (e.g. as a solution or prompt drug-releasing, conventional solid dosage form).

A “modified release dosage form” is one for which the drug release characteristics, time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Pulsed release” or “pulsatile release” refers to an initial release of drug, followed by a period of substantially no release, followed by one or more additional releases of drug separated by a period of substantially no release. This does not mean that there are no blood levels of drugs between periods of release.

Sustained release” or “SR” means the therapeutic pharmaceutical composition is provided in a formulation such that the composition provides an initial therapeutic effect and also an ongoing or additional release of the therapeutic pharmaceutical composition or therapeutic effect over a desired period of time.

Substantially no liver toxicity” means that a patient ingesting a therapeutic pharmaceutical composition consisting essentially of an anticholinergic agent and sedative agent according to embodiments disclosed herein does not experience a substantial increase in liver enzyme production associated with administration of the composition.

### II. Formulations

### A. Potassium Guaiacolsulfonate

Guaiacolsulfonic acid has the structure shown below:

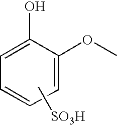

The sulfonic acid group is typically found at the 4 position or the 5 position of the benzene ring. Guaiacolsulfonic acid is usually sold as a salt, for example, potassium guaiacolsulfonate, and may contain a mixture of the 4 and 5-derivatives.

Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic acids.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two: generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md. 2000, p. 704.

For use as a mucolytic agent, the amount of potassium guaiacolsulfonate generally will be the equivalent of about 100 mg/day to about 2,400 mg/day. A typical dosage is about 600 mg to about 1,200 mg administered twice a day.

The preferred dosage ranges for potassium guaiacolsulfonate formulations are:

For IR/SR, 100 to 600 mg IR/600 to 1,200 mg SR administered twice daily. This formulation can also be modified with an enteric coating so that the SR occurs after a two hour delay.

For IR/PR, there are three doses of 100 to 400 mg when dosed twice daily, using a multiparticulate system with delayed release beads.

For any application with sustained release, the dosage will typically be in the range of 300 to 1,200 mg twice daily.

For use as a muscle relaxant, the amount of potassium guaiacolsulfonate generally will be the equivalent of about 600 mg/day to about 3,600 mg/day. A typical dosage is about 300 mg to about 1,800 mg administered twice a day.

The preferred dosage ranges for potassium guaiacolsulfonate formulations are:

For IR/SR, 300 to 600 mg IR/600 to 1,200 mg SR administered twice daily. This formulation can also be modified with an enteric coating so that the SR occurs after a two hour delay.

For IR/PR, there are three doses of 200 to 600 mg when dosed twice daily, using a multiparticulate system with delayed release beads.

For any application with sustained release, the dosage will typically be in the range of 600 to 1,800 mg twice daily.

In a preferred embodiment, the formulation is designed to release potassium guaiacolsulfonate over a 1-24 hr period. In the most preferred embodiment, the total quantity of potassium guaiacolsulfonate is from about 600 mg to about 1200 mg. Preferably, in combinations, the ratio of a total quantity of potassium guaiacolsulfonate to additional drug is from about 1:1 to about 100:1 by weight.

### B. Other Active Agents

Potassium guaiacolsulfonate may be administered as the primary active agent in the substantial absence of other active therapeutic agents, but preferably is administered in combination with other active agents.

The potassium guaiacolsulfonate can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastrointestinal drugs, muscle relaxants, nutritional agents, vitamins, parasympathomimetics, stimulants, anorectics and anti-narcoleptics.

Specific examples of compounds that can be adjunctively administered with potassium guaiacolsulfonate include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydramorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprohamate, mesalamine, metapramine, metaxalone, methadone, methadone, methamphetamine, methocarbamol, methyldopa, methylphenidate, methylsalicylate, methysergid(e), metoclopramide, mianserin, mifepristone, minaprine, mirtazapine, moclobemide, modafomil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindae, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, veniafaxine, viloxazine, vitamin E, zimeldine, ziprasidone, zolmitriptan, zolpidem, zopiclone and isomers, salts, and combinations thereof.

The term “adjunctive administration” as used herein means simultaneous administration of the compounds, in the same dosage form, simultaneous administration in separate dosage forms, and/or separate administration of the compounds.

The most common form of potassium guaiacolsulfonate is in combinations for treatment of bronchial congestion. The antihistamine commonly contained in these combinations is chlorpheniramine. These are usually accompanied by a vasoconstrictor such as phenylephrine or pseudoephedrine which is useful in drying the nasal passages. Other preferred combinations include dextromethorphan hydrobromide, codeine, hydrocodone, phenylephrine hydrochloride, phenylpropanolamine hydrochloride, pseudoephedrine hydrochloride, ephedrine, chlorpheniramine maleate, brompheniramine maleate, phenindamine tartrate, pyrilamine maleate, doxylamine succinate, phenyltoloxamine citrate, diphenhydramine hydrochloride, promethazine, clemastine fumerate, aspirin, ibuprofen, acetaminophen, naprosin, or combinations thereof.

### C. Excipients

Formulations are prepared using pharmaceutically acceptable “carriers” composed of materials that are considered safe and effective and may be administered to an individual without causing undesirable biological side effects or unwanted interactions. The term “carrier” refers to all components present in the pharmaceutical formulation other than the active ingredient or active ingredients. The term “carrier” includes but is not limited to diluents, binders, lubricants, disintegrators, fillers, and coating compositions. The term “carrier” also includes all components of the coating composition, which may include plasticizers, pigments, colorants, stabilizing agents, and glidants.

The delayed release dosage formulations may be prepared as described in references such as “Pharmaceutical dosage form tablets”, Eds. Liberman et. a. (New York, Marcel Dekker, Inc., 1989), “Remington—The science and practice of pharmacy”, 20th Ed., Lippincott (Williams & Wilkins, Baltimore, Md., 2000), and “Pharmaceutical dosage forms and drug delivery systems”, 6th Ed., Ansel et.al., (Media, Pa.: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT.®. (Roth Pharma, Westerstadt, Germany), zein, shellac, and polysaccharides. The coating material may contain conventional excipients, such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “filers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone.®. XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis(2-ethylthioxy)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER.®. 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, capsules, beads, granules or particles may also contain minor amount of nontoxic auxiliary substances such as wetting or emulsifying agents, dyes, pH-buffering agents, and preservatives.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol manostearates may also be used. Pigments such as titanium dioxide may also be used. Small quantities of an anti-foaming agent, such as a silicone (e.g., simethicone), may also be added to the coating composition.

Formulations may also include the high functionality tableting excipient, silicified microcrystalline cellulose, (e.g. ProSolv.®. HD-90; ProSolv SMCC 50; ProSolv SMCC 90; JRS Pharma, Patterson, N.Y.) with high compactibility, high intrinsic flow, enhanced lubrication efficiency and improved blending properties.

Formulations may include additional excipients that can enhance the rate and extent of oral absorption of potassium guaiacolsulfonate. Preferably, the formulation includes one or more absorption enhancers that increase the rate of the absorption of potassium guaiacolsulfonate across the buccal or intestinal mucosa, as compared to the same formulation in the absence of the absorption enhancer(s). Suitable absorption enhancers include, but are not limited to, surfactants, such as anionic and non-ionic surfactants; phospholipids; fatty acids, such as capric acid, and salts thereof; fatty acid glycerides; bile acids, such as cholic acid and deoxycholic acid; amino acids; mixed micelles; oil-in-water emulsions; chelating agents, such as EDTA and EGTA; glycyrrhizic acid; cyclodextrins, such as hydroxypropyl-beta-cyclodextrin; polysaccharides, such as chitosans; liposaccharides; and ammonium glycerizinate.

### D. Dosage Forms

Formulations with different drug release mechanisms described above may be combined in a final dosage form including single or multiple units. Examples of multiple units include multilayer tablets or capsules containing tablets, beads, or granules in a solid or liquid form.

### Immediate Release Formulations

Typical immediate release formulations include compressed tablets, gels, films, coatings, liquids and particles that can be encapsulated, for example, in a gelatin capsule. Methods for preparing coatings, covering or incorporating drugs, are known in the art.

The immediate release dosage unit of the dosage form, i.e., a tablet, a plurality of drug-containing beads, granules or particles, or an outer layer of a coated core dosage form, contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads). A preferred method for preparing immediate release tablets (e.g., as incorporated into a capsule) is by compressing a drug-containing blend, e.g., blend of granules, prepared using a direct blend, wet-granulation or dry-granulation process. Immediate release tablets may also be molded rather than compressed, starting with a moist material containing a suitable water-soluble lubricant. However, preferred tablets described herein are manufactured using compression rather than molding. A preferred method for forming an immediate release drug-containing blend is to mix drug particles directly with one or more excipients such as diluents (or fillers), binders, disintegrants, lubricants, glidants, and/or colorants. As an alternative to direct blending, a drug-containing blend may be prepared by using a wet-granulation or dry-granulation process. Beads containing the active agent may also be prepared by any one of a number of conventional techniques, typically starting from a fluid dispersion. For example, a typical method for preparing drug-containing beads involves blending the active agent with conventional pharmaceutical excipients such as microcrystalline cellulose, starch, polyvinylpyrrolidone, methylcellulose, talc, metallic stearates, and/or silicone dioxide. The admixture is used to coat a bead core such as a sugar sphere (e.g., a “non-pareil”) having a size of approximately 20 to 60 mesh.

An alternative procedure for preparing drug beads is by blending the drug with one or more pharmaceutically acceptable excipients, such as microcrystalline cellulose, lactose, cellulose, polyvinyl pyrrolidine, talc, magnesium stearate, and/or a disintegrant, extruding the blend, spheronizing the extrudate, drying and optionally coating the bead to form immediate release beads.

### Extended or Sustained Release Dosage Forms

Extended release formulations are generally prepared as diffusion or osmotic systems, for example, as described in “Remington—The science and practice of pharmacy”, 20th Ed., Lippincott (Williams & Wilkins, Baltimore, Md., 2000). A diffusion system typically consists of one of two types of devices, reservoir and matrix, which are well-known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and Carbopol.®. 934, and polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeability and high permeability coating materials in suitable proportion.

An immediate release portion can be added to the extended release system by means of either applying an immediate release layer on top of the extended release core using coating or compression processes or in a multiple unit system such as a capsule containing extended and immediate release beads.

Extended release tablets containing hydrophilic polymers are prepared by techniques commonly known in the art such as direct compression, wet granulation, or dry granulation processes. These formulations usually incorporate polymers, diluents, binders, and lubricants as well as the active pharmaceutical ingredient. The usual diluents include inert powdered substances such as different kinds of starch; powdered cellulose, especially crystalline and microcrystalline cellulose; sugars such as fructose, mannitol and sucrose; grain flours; and similar edible powders. Typical diluents include, for example, various types of starch, lactose, mannitol, kaolin, calcium phosphate or sulfate; inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates; methylcellulose; and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### Delayed Release Dosage Forms

Delayed release dosage formulations are created by coating a solid dosage form with a film of a polymer which is insoluble in the acid environment of the stomach, but soluble in the neutral environment of the small intestine.

The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolysable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT.®. (Rohm Pharma: Westerstadt, Germany), including EUDRAGIT.®. L30D-55 and L100-55 (soluble at pH 5.5 and above), EUDRAGIT.®. L-100 (soluble at pH 6.0 and above), EUDRAGIT.®. S (soluble at pH 7.0 and above, as a result of a higher degree of esterification), and EUDRAGITs.®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, and glidants. A plasticizer is normally present to reduce the fragility of the coating, and will generally represent about 10 wt % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Alternatively, a delayed release tablet may be formulated by dispersing the drug within a matrix of a suitable material such as a hydrophilic polymer or a fatty compound. Suitable hydrophilic polymers include, but are not limited to, polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

### Pulsed Release Dosage Forms

A pulsed release dosage form is one that mimics a multiple dosing profile without repeated dosing and typically allows at least a twofold reduction in dosing frequency as compared to the drug presented as a conventional dosage form (e.g. as a solution or prompt drug-releasing, conventional solid dosage form). A pulsed release profile is characterized by a time period of no release (lag time) or reduced release followed by rapid drug release.

Each dosage form contains a therapeutically effective amount of active agent. In one embodiment of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 30 wt. %, preferably 60wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

For dosage forms mimicking a three times daily dosing profile, approximately 25 wt. % to 40 wt. % of the total amount of active agent in the dosage form is released in the initial pulse, and approximately 25 wt. % to 40 wt. % of the total amount of active agent in the dosage form is released in each of the second and third pulses. For dosage forms that mimic a three times daily dosing profile, release of the second pulse preferably takes place approximately 3 hours to 10 hours, and more preferably approximately 4 to 9 hours, following oral administration. Release of the third pulse occurs about 2 hours to about 8 hours following the second pulse, which is typically about 5 hours to approximately 18 hours following oral administration.

The dosage form can be a closed capsule housing at least two drug-containing dosage units, each dosage unit containing one or more compressed tablets, or may contain a plurality of beads, granules or particles, providing that each dosage unit has a different drug release profile. The immediate release dosage unit releases drug substantially immediately following oral administration to provide an initial dose. The delayed release dosage unit releases drug approximately 3 hours to 14 hours following oral administration to provide a second dose. Finally, an optional second delayed release dosage unit releases drug about 2 hours to 8 hours following the release of the second dose, which is typically 5 hours to 18 hours following oral administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of heads, granules or particles that release drug substantially immediately following oral administration to provide an initial dose. The delayed release dosage unit contains a plurality of coated beads or granules, which release drug approximately 3 hours to 14 hours following oral administration to provide a second dose.

An optional second delayed release dosage unit contains coated beads or granules that release drug about 2 to 8 hours following administration of the initial delayed release dose, which is typically 5 to 18 hours following oral administration. The beads or granules in the delayed release dosage unit(s) are coated with a bioerodible polymeric material. This coating prevents the drug from being released until the appropriate time, i.e., approximately 3 hours to less than 14 hours following oral administration for the delayed release dosage unit and at least 5 hours to approximately 18 hours following oral administration for the optional second delayed release dosage unit. In this dosage form the components may be admixed in the tablet or may be layered to form a laminated tablet.

Another dosage form is a tablet having a drug-containing immediate release dosage unit, a delayed release dosage unit, and an optional second delayed release dosage unit, wherein the immediate release dosage unit contains an outer layer that releases the drug substantially immediately following oral administration. The arrangement of the remaining delayed release dosage(s), however, depends upon whether the dosage form is designed to mimic twice daily dosing or three times daily dosing.

In the dosage form mimicking twice daily dosing, the delayed release dosage unit contains an inner core that is coated, with a bioerodible polymeric material. The coating is applied such that release of the drug occurs approximately 3 hours to less than 14 hours following oral administration. In this form, the outer layer completely surrounds the inner core.

In the dosage form mimicking three times a day dosing, the (first) delayed release dose contains an internal layer that releases drug approximately 3 hours to less than 14 hours following oral administration. This internal layer is surrounded by the outer layer. The second delayed release dosage unit generally contains an inner core that releases the drug at least 5 hours to approximately 18 hours following oral administration. Thus, the layers of this tablet (starting from the external surface) contain an outer layer, an internal layer and an inner core. The inner core contains delayed release beads or granules. Furthermore, the internal layer contains the drug coated with a bioerodible polymeric material. Alternatively, in this particular dosage form mimicking three times a day dosing, both the delayed release dosage unit and second delayed release dosage units are surrounded by an inner layer. This inner layer is free of active agent. Thus, the layers of this tablet (starting from the external surface) contain an outer layer, inner layer and an admixture of the delayed release dosage units. The first delayed release pulse occurs once the inner layer is substantially eroded thereby releasing the admixture of the delayed release dosage units. The dose corresponding to the (first) delayed release dosage unit is released immediately since the inner layer has prevented access to this dose for the appropriate time, e.g., from approximately 3 hours to 10 hours. The second delayed release dose, however, is formulated to effectively delay release for at least 5 hours to approximately 18 hours following oral administration.

For formulations mimicking twice daily dosing, it is preferred that the delayed release dose is released approximately 3 hours to up to 14 hours, more preferably approximately 5 hours to up to 12 hours, following oral administration. For formulations mimicking three times daily dosing, it is preferred that the (first) delayed release dose is released approximately 3 to 10 hours, preferably 4 hours to 9 hours, following oral administration. For dosage forms containing a third dose, the third dose (i.e., the second delayed release dose) is released at least 5 hours to approximately 18 hours following oral administration.

In still another embodiment, a dosage form is provided which contains a coated core-type delivery system wherein the outer layer is an immediate release dosage unit containing an active agent, such that the active agent therein is immediately released following oral administration; an intermediate layer there under which surrounds a core; and a core which contains immediate release beads or granules and delayed release beads or granules, such that the second dose is provided by the immediate release beads or granules and the third dose is provided by the delayed release beads or granules.

Drug complexes are generally prepared by complexing the drug with a pharmaceutically acceptable ion-exchange resin. The complex is formed by reaction of a functional group of the drug with a functional group on the ion exchange resin. Drug is released by exchanging with appropriately charged ions within the gastrointestinal tract.

### Ion-Exchange Resins

Ion-exchange resins are water-insoluble, cross-linked polymers containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can also be, e.g., silica gel modified by the addition of ionic groups. The covalently bound salt forming groups may be strongly acidic (e.g., sulfonic acid or sulfuric acid) or weakly acidic (e.g., carboxylic acid). In general, those types of ion-exchangers suitable for use in ion-exchange chromatography and for such applications as deionization of water are suitable for use in these controlled release drug preparations. Such ion-exchangers are described by H. F. Walton, in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975).

Resins include Amberlite.®. IRP-69 (Rohm and Haas) INDION.®. 224, INDION.®. 244, and INDION.®. 254 (Ion Exchange (India) Ltd.). These resins are sulfonated polymers composed of polystyrene cross-linked with divinylbenzene. Any ion-exchange resins currently available and those that should become pharmaceutically acceptable and available in the future can also be used. Commercial sources of ion exchange resins that are either pharmaceutically acceptable or may become pharmaceutically acceptable in the future include, but are not limited to, Rohm and Haas, The Dow Chemical Company, and Ion Exchange (India) Ltd.

The size of the ion-exchange particles should be less than about 2 millimeter, more preferably below about 1000 micron, more preferably below about 500 micron, and most preferably below about 150 micron. Commercially available ion-exchange resins (Amberlite.®. IRP-69, INDION.®. 244 and INDION.®. 254) have a particle size range less than 150 microns.

Drug is bound to the resin by exposure of the resin to the drug in solution via a batch or continuous process (such as in a chromatographic column). The drug-resin complex thus formed is collected by filtration and washed with an appropriate solvent to insure removal of any unbound drug or by-products. The complexes are usually air-dried in trays. Such processes are described in, for example, U.S. Pat. Nos. 4,221,778, 4,894,239, and 4,996,647.

Binding of drug to resin can be accomplished according to four general reactions. In the ease of a basic drug, these are: (a) resin (Ha-form) plus drug (salt form); (h) resin (Na-form) plus drug (as free base); (e) resin (H-form) plus drug (salt form): and (d) resin (H-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

The resin-drug complexes can be incorporated into tablets, capsules, beads, films, coatings or particles. The resin-drug complexes or particles containing the complexes can also be suspended in a liquid such as a syrup. The complexes or particles can also be coated with a material such as art enteric coating or barrier to alter release properties. Complexes with different coatings, or mixture of uncoated with coated complexes or particles, can be used to create mixtures with different release properties.

### III. Dosage Unit Packs

Kits are provided wherein the dosage form is packaged to provide a method to conveniently begin dose titration at lower doses, for example, beginning at 200 mg, gradually increasing to 600 mg, 1,200 mg, 1,800 mg, and/or 2,400 mg, over a period ranging from three days up to 16 weeks. For the treatment of fibromyalgia, the dose titration would start at 600 mg increasing up to 3,600 mg, over a period ranging from three days to 16 weeks.

The packaging material may be a box, bottle, blister package, tray, or card. The kit may include a package insert instructing the patient to take a specific dose at a specific time, for example, a first dose on day one, a second higher dose on day two, a third higher dose on day three, and so on, until a maintenance dose is reached. Alternatively, the dose unit pack may contain multiple formulations designed to give different potassium guaiacolsulfonate doses and/or different drug combinations, one of which includes potassium guaiacolsulfonate that can be taken at different times, e.g. on different days or different times of the day.

### IV. Methods of Manufacturing

As will be appreciated by those skilled in the art and as described in the pertinent texts and literature, a number of methods are available for preparing drug-containing tablets, beads, capsules, granules or particles, films and coatings that provide a variety of drug release profiles. Such methods include, but are not limited to, coating a drug or drug-containing composition with an appropriate coating material, increasing drug particle size, placing the drug within a matrix of excipient and other fillers, coating the material with an enteric coating, and forming complexes of the drug with a suitable complexing agent such as an ion-exchange resin.

Coatings can be applied as aqueous or organic solutions or suspensions. Film coatings are typically thin barrier films, providing protection or color to the particles or tablets. Active ingredient can be incorporated into the coating. Coatings may be formed of lipid or by hot melting of polymers. This provides coatings of between 25 and several hundred microns in thickness. These coatings protect against moisture. No evaporation of solvents is required. Sugar coatings are generally between 0.5 and 2 mm. These are used to provide taste masking and sealing, as well as for protection and coating of temperature-sensitive and fragile products. The coating is applied by spraying of a syrup onto the particles.

These sprayed coatings can vary between approximately 5 microns and 50 microns or more. Coatings can be applied as polymeric solutions or sprays by fluidized bed reactors, by spray coating (top spray, Wurster coating—bottom spray), or tangential spray—rotor pellet coating), or drum or pan coaters. Top spray coatings are used for general coatings including enteric coatings. Particles are fluidized in the flow of heated air, which is introduced into the product container and the coating liquid is sprayed into the fluid bed from above. Drying takes place as the particles move upward. Bottom spraying is particularly suitable for controlled release of active ingredients. In the Wurster process, a complete sealing of the surface can be achieved with a low usage of coating substance. The spray nozzle is fitted in the base plate resulting in a spray pattern that is concurrent with the air feed. By using a Wurster cylinder and a base plate with different perforations, the particles to be coated are accelerated inside the Wurster tube and fed through the spray cone concurrently. As the particles continue traveling upwards, they dry and fall outside the Wurster tube back towards the base plate. They are guided from the outside back to the inside of the tube where they are once again accelerated by the spray. This produces an extremely even film. Particles of different sizes are evenly coated. Particularly suitable for protective coatings/color coatings where the product throughput rates are high. For continuous fluid bed coatings, the product is continuously fed into one side of the machine and is transported onwards via the sieve bottom by means of the air flow. Depending on the application, the system is sub-divided into pre-heating zones, spray zones and drying zones whereby spraying can take place from below in the form of a bottom spray. The dry, coated particle's are continuously extracted. Tangential spray coatings (Rotor pellet coating) are ideal for coatings with high solid content. The product is set into a spiral motion by means of a rotating base plate, which has air fed into the powder bed at its edge. The spray nozzle is arranged tangentially to the rotor disc and also sprays concurrently into the powder bed. Very thick film layers can be applied by means of the rotor method. Tablets and dragees are coated using drum or pan coating. These are typically for the application of protective films or taste masking.

Powder particles can be agglomerated in a fluid bed to build up powder granulates, typically in the size range of 0.2 and 2.5 mm. The powder is moistened in order to form liquid bridges between the particles. The spray liquid can be either water or an organic solvent which dissolves the powder or a binder. The moistened granulates are dried and cooled. These have a low bulk density and are highly wafer soluble. Wet granulation is used to build up granulates from powder. These are generally denser and more mechanically stable particles than fluid bed granulates. These produce grains between 0.1 and 10 mm. Wet granulation in a vertical granulator is the classical method for building up granulates from powder. In this process, powder is fed to a product container and then moistened or sprayed with molten material in order to increase the cohesive forces. The liquid can be water or an organic solvent, if necessary with a binder. At the same time, the ingredients are mixed together vigorously. Denser granulates are formed than in the case of in the fluid bed. The products are highly suitable for making into tablets, compact, with low hygroscopicity. Spray granulation is the drying of liquids (solutions, suspensions, melts) while simultaneously building up granulates.

Germs can be provided for granulates (foreign germs) or can form in the fluid bed due to abrasion and fracture (inherent germs). The spray liquid coats the germs and is then dried. Spray granulates are denser and harder in comparison with agglomerates. The spray granulation of different starting materials that have been mixed in the liquid phase produces granulates, in which the starting materials are very evenly distributed. If the process is set up correctly, liquids can also be encapsulated in a fixed matrix in this way.

If the matrix material is dissolved in the liquid phase, the granulates are made by means of spray granulation. If the matrix material is present in the form of powder, the granulates are made by means of wet granulation. This encapsulation process is mainly applied in the food industry. If necessary, a protective coating can be applied to the spray granulates in an additional step.

Blending is the dry mixing of ingredient to produce a uniform distribution of components. In solid processes, various individual products of different density and concentration and in different amounts are often admixed to form a homogeneous mixture. In the pharmaceutical area, very different quantities and proportions of active and auxiliary ingredients (corn starch, lactose, PVP, etc.) are mixed together. Specific auxiliary materials such as lubricants or flavorings may also be added. Mixing may be necessary in different process sections. For instance, compression aids, flow controlling media and external phases are added following the granulation process and before compression.

Direct pelletizing is the manufacture of pellets directly from powder. Pellets can be prepared by building up layer by layer around a core, or a round pellet can be extruded by spheronizing. Spray granulation can also be used for the build-up of liquid particles. In direct pelletizing, pellets are manufactured directly from powder with a binder or solvent. This is a fast process and yields compact, round pellets, which have a higher density than spray granulates and agglomerates. Pellet diameters are between 0.2 and 1.2 mm. Pellets can be made into tablets or used to fill capsules. Pelletizing by layering results in the layer by layer build-up of material around a core. This is ideal for forming round pellets with separate layers of powder coatings and/or active agent. The layers are densely applied due to the movement of the pellets in the rotor. Thick layers can be applied to the starting grains, which allow large amounts of active to be incorporated. These have a higher density than spray granulates and agglomerates. Typical diameters are between 0.6 and 2.5 mm. In spheronizing, round pellets are formed from irregular wet granulates and extruded products. The moist granulates or extruded products are fed onto a rotating/pelletizing plate. The surface is smoothed due to the intensive rolling movement and spherical pellets are produced due to the intensive rolling movement. This results in narrow particle size distribution and good flow behavior. Pellets have a higher density than spray granulates and agglomerates. Typical particle diameters are between 0.5 and 2.5 mm. Spray granulation is the drying of liquids (solutions, suspensions, melts) while simultaneously building up of granulates. These are denser and harder than agglomerates and have a size between 0.2 and 5 mm.

For detailed information. concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6th Ed. (Media, Pa.: Williams & Wilkins, 1995). A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrates, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (e.g., “non-pareil”) having a size of approximately 20 to 60 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### V. Methods of Administration

The amount of potassium guaiacolsulfonate and the type (time and rate) of release in the compositions or pharmaceutical formulations administered to a patient may vary depending upon multiple factors including, but not limited to, the disorder to be treated, the particular composition to be administered, the patient's degree of illness, the patient's weight, and the patient's age.

In a preferred embodiment, the potassium guaiacolsulfonate formulations are used in cold and cold/allergy formulations as a mucolytic agent for the treatment of bronchial congestion and coughs associated with the common cold, asthma, and other respiratory illnesses (e.g., sinusitis, pharyngitis, bronchitis), treatment of allergic rhinitis, sinusitis, and the common cold.

In another preferred embodiment, the formulations are used to treat one or more symptoms of fibromyalgia. In additional embodiments, the formulations may be used for the treatment of pain and Irritable Bowel Syndrome. In one embodiment, the guaiacolsulfonate is administered to an individual to induce muscle relaxation in a dosage equivalent to between about 600 mg/day and about 3,600 mg/day.

The formulations may be administered to an individual for relief of one or more symptoms associated with a disorder such as sleep disorders, hypothyroidism, Lyme disease, chronic myofascial pain, fibromyalgia, hormonal imbalances, and pain resulting from injuries or chronic pain from any source, local or generalized, for example, where the drug is administered to an individual with back pain, pelvic pain, Ehlers-Danlos Syndrome or restless leg syndrome. In one embodiment, the guaiacolsulfonate is administered to an individual to potentiate the effects of pain analgesics, where the analgesic is paracetamol, morphine, opiates, doxepin, naprosin, aspirin, ibuprofen, or acetaminophen.

Modifications and variations will be apparent to those skilled in the art and are intended to be encompassed by the following claims. All publications cited herein are incorporated by reference.

What is claimed is:

1. A multi-phase guaiacolsulfonate salt formulation.

2. The formulation of claim 1, wherein the salt is potassium.

3. The formulation of claim 1 further comprising one or more additional active agents.

4. The formulation of claim 3, wherein the additional active agent is selected from the group consisting of expectorants, antitussives, decongestants, antihistamines, and analgesics.

5. The formulation of claim 4, wherein the additional active agent is selected from the group consisting of dextromethorphan hydrobromide, codeine, hydrocodone, phenylephrine hydrochloride, phenylpropanolamine hydrochloride, pseudoephedrine hydrochloride, ephedrine, chlorpheniramine maleate, brompheniramine maleate, phenindamine tartrate, pyrilamine maleate, doxylamine succinate, phenyltoloxamine citrate, diphenhydramine hydrochloride, promethazine, clemastine fumerate, aspirin, ibuprofen, acetaminophen, naprosin, potassium, guaiacolsulfonate and combinations thereof.

6. The formulation of claim 5, wherein the additional active agent is guaifenesin, bromohexidine, theobromine or combinations thereof.

7. The formulation of claim 1 comprising an immediate release guaiacolsulfonate component.

8. The formulation of claim 1 comprising a delayed release guaiacolsulfonate component.

9. The formulation of claim 1 comprising a sustained release guaiacolsulfonate component.

10. The formulation of claim 1 comprising a pulsed release guaiacolsulfonate component.

11. The formulation of claim 1 comprising an immediate release and a delayed, sustained or pulsed guaiacolsulfonate component.

12. The formulation of claim 11 comprising a bilayer tablet containing an immediate release component of potassium guaiacolsulfonate in one layer and a sustained release and/or delayed release component of potassium guaiacolsulfonate in the second layer.

13. The formulation of claim 1, wherein the formulation is designed to release guaiacolsulfonate over a 1-24 hr period.

14. The formulation of claim 1, wherein a total quantity of guaiacolsulfonate is from about 600 mg to about 3600 mg,

15. The formulation of claim 1, wherein a total quantity of guaiacolsulfonate is from about 100 mg/day to about 2,400 mg/day.

16. The formulation of claim 11 for immediate release of about 100 to 600 mg guaiacolsulfonate and sustained release of about 600 to 1200 mg guaiacolsulfonate.

17. The formulation of claim 10 comprising pulsed release of three doses of 100 to 400 mg in a multiparticulate system with delayed release beads.

18. The formulation of claim 3, wherein a ratio of a total quantity of potassium guaiacolsulfonate to the additional active agent is from about 1:1 to about 100:1 by weight.

19. The formulation of claim 3, wherein the release of one or more additional active agents may be immediate, sustained, delayed release or combinations thereof.

20. The formulation of claim 1 in a package of individual unit dosage forms providing different dosages of guaiacolsulfonate.

21. The formulation of claim 1 in a package of individual unit dosage forms marked with instructions providing an administration regime for different dosages of guaiacolsulfonate.

22. The formulation of claim 1 in a package of individual unit dosage forms providing multiple formulations that contain different guaiacolsulfonate doses or different drug combinations, one of which includes guaiacolsulfonate, that can be taken at different times on different days or different times of the day.

23. The formulation of claim 22 wherein the regime provides for an escalating dosage.

24. The formulation of claim 1, wherein the dosage unit form is selected from the group consisting of tablets, gels, liquids, capsules, beads, microparticles, films, lozenges, and sublingual tablets.

25. The formulation of claim 24 comprising a mixture of immediate, delayed or sustained release granules compressed into a solid matrix tablet, a rapidly disintegrating tablet, or filled into a capsule.

26. The formulation of claim 1, wherein the solid matrix tablet contains silicified microcrystalline cellulose

27. The formulation of claim 1, wherein the dosage form is a system, designed to achieve absorption of guaiacolsulfonate through the buccal cavity.

28. The formulation of claim 27, wherein, the dosage form further comprises an absorption enhancer designed to increase the bioavailability of guaiacolsulfonate across the mucosa.

29. A method of administering guaiacolsulfonate comprising administering the formulation of claim 1.

30. The method of claim 29, wherein the guaiacolsulfonate is administered to an individual for relief of one or more symptoms associated with a disorder selected from the group consisting of sleep disorders, hypothyroidism, lyme disease, chronic myofascial pain, fibromyalgia, and hormonal imbalances.

31. The method of claim 29, wherein the guaiacolsulfonate is administered to an individual with pain, including back pain, pelvic pain, and Ehlers-Danlos Syndrome.

32. The method of claim 29, wherein the guaiacolsulfonate is administered to an individual with restless leg syndrome.

33. The method of claim 29, wherein the guaiacolsulfonate is administered to an individual to induce muscle relaxation in a dosage equivalent to between about 600 mg/day and about 3,600 mg/day.

34. The method of claim 29, wherein the guaiacolsulfonate is administered to an individual with Irritable Bowel Syndrome.

35. The method of claim 29, wherein the guaiacolsulfonate is administered to an individual to potentiate the effects of pain analgesics, where the analgesic is selected from the group consisting of paracetamol, morphine, opiates, doxepin, naprosin, aspirin, ibuprofen, acetaminophen.

36. The method of claim 29, wherein the guaiacolsulfonate is administered to treat coughing and symptoms or diseases associated with coughing.

37. The method of claim 29, wherein the guaiacolsulfonate is packaged in a unified medication package comprising a multiplicity of medications, functional indicia, and instructions for coordinating the medications together as a regimen.

38. The method of claim 29 comprising providing medication dosages in blister packs or pouches, organized into event modules associated with particular daily events, and having corresponding event indicia.

39. The method of claim 38, wherein the event modules are further organized into day modules having day indicia.

40. The method of claim 38, wherein the event modules are organized into compartments of a box.
